(19) United States
(12) Patent Application Publication
(10) Pub. No.: US 2026/0232543 A1
(43) Pub. Date: Aug. 13, 2026

(54) KERATIN BOND DISSOLUTION FOR KERATIN-BASED ADHESIVE BONDS

(71) Applicants: Jonathan Sexsmith, Stuart, FL (US); Whitney Sexsmith, Stuart, FL (US); Twee Le-Roth, Simi Valley, CA (US)

(72) Inventors: Jonathan Sexsmith, Stuart, FL (US); Whitney Sexsmith, Stuart, FL (US); Twee Le-Roth, Simi Valley, CA (US)

(73) Assignee: Ubertas International INC, Stuart, FL (US)

(21) Appl. No.: 19/536,883

(22) Filed: Feb. 11, 2026

Related U.S. Application Data
(60) Provisional application No. 63/757,132, filed on Feb. 11, 2025.

(51) Int. Cl. A61Q 5/00; A61Q 5/04; A61Q 5/06

(52) U.S. Cl. CPC A61K 8/00; A61Q 5/00; A61Q 5/04; A61Q 5/06; A61K 2300/00; A61K 2800/882

(57) ABSTRACT

Provided herein are one or more compositions that may at least partially remove and/or dissolve keratin-based adhesive bonds, one or more methods of applying the one or more compositions, and one or more methods of making the one or more compositions. The one or more compositions may include one or more active ingredients such as one or more proteolytic enzymes. The one or more compositions may be applied as part of a process of removing one or more keratin bond hair extensions from hair.

### CROSS-REFERENCE TO RELATED APPLICATIONS

This application is a U.S. non-provisional application and claims the benefit of U.S. provisional application umber 63/757,132, filed Feb. 11, 2025, and entitled “Keratin Bond Dissolution for Hair Extension Removal”. The above-mentioned provisional application is incorporated herein by reference in its entirety.

### FIELD

This disclosure relates generally to hair care products, cosmetic technology, and/or medical adhesive systems, and methods of making and applying one or more hair care products, cosmetic technology, biomedical engineering systems, and/or medical adhesive systems. More specifically, this disclosure relates to one or more compositions that may at least partially remove and/or dissolve keratin-based adhesive bonds, one or more methods of applying the one or more compositions, and one or more methods of making the one or more compositions.

### BACKGROUND

Keratin-based adhesives have wide uses including many uses for hair care, cosmetic technologies, medical adhesive systems, and the like. As one particular example, keratin-based adhesives are sometimes used to bond, or fuse, hair extensions to a person's hair. Hair extensions, generally, are a popular cosmetic technology that allow people to transform their hairstyle in numerous ways. Keratin bond hair extensions (also sometimes referred to as fusion hair extensions, among other names) are one type of hair extension available on the market today and, by use of keratin-based adhesives, are known to provide a durable bond to a person's hair. For example, as compared to tape hair extensions, which are another type of hair extension and which last a few weeks in a person's hair (e.g., 6 weeks), keratin bond hair extensions can last many months in a person's hair (e.g., 6 months). Keratin bond hair extensions may be applied by heating a keratin-based adhesive (e.g., one or more keratin gel beads) until it melts and then letting the melted keratin-based adhesive cool to a hardened state. As the melted keratin-based adhesive cools to the hardened state, it adheres, bonds, or fuses, with any of the person's hair and any hair extension strands that the now-cooling keratin-based adhesive is in contact with. Thus, this process of heating and cooling keratin-based adhesive results in a keratin bond hair extension that is adhered, bonded, or fused, to a person's hair.

Due to the bond between the keratin bond hair extension and a person's hair that is provided by the keratin-based adhesive, removing the keratin bond hair extension can be difficult. Indeed, existing methods of removing a keratin bond hair extension can be cumbersome, labor intensive and/or time-consuming. For example, existing methods of removing a keratin bond hair extension may require a second person to at least assist in locating the keratin bonds and, depending on the number of hair extensions bonded to a person's hair, may require upwards of a few hours to successfully remove all hair extensions from the person's hair. One existing method of removing a keratin bond hair extension may involve another person locating where a keratin bond exists in a person's hair, rubbing the keratin bond with ethyl alcohol (e.g., an ethyl alcohol solution with a 70% or greater concentration of ethyl alcohol), breaking the keratin bond with pliers, pulling on the area with the keratin bond with the pliers, and repeating those steps on the same or different keratin bonds until the keratin bond hair extension can be removed. Pulling on the keratin bond with the pliers often results in the person's hair breaking and/or being pulled with the keratin bond hair extension. To remove a single hair extension, this existing process may take around 20 minutes. Moreover, this existing process would need to be repeated for each keratin bond hair extension that is bonded to the person's hair. Such an existing process, as noted above, is cumbersome (e.g., may require at least two people), labor intensive (e.g., pliers may need to be used on each bond to assist in breaking the bond), and time-consuming (e.g., may take plural hours to completely remove all hair extensions).

### SUMMARY

Described herein are one or more compositions that may at least partially remove and/or dissolve keratin-based adhesive bonds, one or more methods of applying the one or more compositions, and one or more methods of making the one or more compositions. For example, some aspects described herein relate to one or more compositions that may at least partially remove and/or dissolve keratin-based adhesive bonds. The one or more compositions may be embodied as, used as part of, and/or applied to numerous different technologies, products, and/or processes that have a need to at least partially remove and/or dissolve keratin-based adhesives. Example technologies, products, and/or processes may include hair care, or beauty, products; cosmetic technologies; biomedical engineering systems, medical adhesive systems; and the like. In general, the aspects described herein may generally improve the ways in which keratin-based adhesives may be dissolved and/or removed in any bonding application.

As one particular example, described herein are one or more compositions that may at least partially remove and/or dissolve keratin-based adhesive bonds to assist in the removal of one or more keratin bond hair extensions from a person's hair. The one or more compositions described herein may exhibit various improvements over the cumbersome, time-consuming, and existing processes for removing keratin bond hair extensions mentioned above in the Background section. Indeed, with the global hair care and hair extension market showing large growth (e.g., some projections expect the market to exceed $5 billion by 2027), there is an ever-present need to improve compositions that are used to remove keratin bond hair extensions in, for example, their ease of use and general performance. In some arrangements and based on one or more aspects described herein, keratin bond hair extensions may be removed from a person's hair by themselves (e.g., without a second person assisting in removing the hair extensions), by their fingers and/or hands (e.g., without using pliers to aid in the breaking of adhesive bonds, pulling on the adhesive bonds, and breaking the person's hair), and/or within minutes (e.g., a single hair extension can be removed within 1.5-3 minutes).

This disclosure also describes additional ways in which hair care and/or hair extension removal can be improved. For example, described herein are one or more compositions that at least partially remove and/or dissolve keratin-based adhesive bonds while preventing hair damage, scalp irritation, and other inefficiencies common with existing processes of removing keratin bond hair extensions (e.g., hair damage and/or scalp damage caused by pliers and/or ethyl alcohol, as used in existing processes of removing keratin bond hair extensions). As another example, described herein are one or more compositions that at least partially remove and/or dissolve keratin-based adhesive bonds by using, or otherwise including, ingredients that are non-toxic, or otherwise less toxic, and/or biodegradable as compared to ingredients used as part of existing processes of removing keratin bond hair extensions (e.g., the one or more compositions may not use or otherwise include ethyl alcohol, mineral oils, and the like). As yet another example, described herein are one or more compositions that at least partially remove and/or dissolve keratin-based adhesive bonds by use of ingredients that are plant-derived, sustainable and/or renewable as compared to ingredients used as part of existing processes of removing keratin bond hair extensions.

It will be appreciated that any of the variations, aspects, features and options described in view of the systems, methods, and/or compositions apply equally to the systems, methods, and/or compositions, and vice versa. It will also be clear that any one or more of the above variations, aspects, embodiments, features, and options can be combined.

Additional advantages will be apparent from the following detailed description. The examples and descriptions herein are to be regarded as illustrative in nature and not restrictive.

### BRIEF DESCRIPTION OF THE DRAWINGS

This disclosure will now be described, by way of example, with reference to the accompanying drawings, in which:

FIG. 1 depicts an example method of making a composition to assist in removing and/or dissolving one or more keratin-based adhesive bonds in accordance with one or more embodiments described herein.

FIG. 2 depicts an example method of applying a composition to assist in removing and/or dissolving one or more keratin-based adhesive bonds in accordance with one or more embodiments described herein.

FIGS. 3A-3E depict example illustrations of applying a composition to assist in removing and/or dissolving one or more keratin-based adhesive bonds in accordance with one or more embodiments described herein.

### DETAILED DESCRIPTION

Reference will now be made in detail to implementations and embodiments of various aspects and variations of compositions and methods disclosed herein. Various examples of compositions and methods are described herein. Although several example variations of the compositions and methods are described herein, it is to be understood that other variations may be utilized, and additional structural and/or functional modifications may be made without departing from the scope described herein. Various aspects are capable of other implementations and of being practiced or being carried out in various different ways. Therefore, this disclosure should not be construed as being solely limited to the examples set forth herein. Rather, the example aspects and embodiments described herein are provided so that this disclosure will be thorough and complete, and will fully convey example implementations.

Some aspects described herein relate to one or more compositions that at least partially remove and/or dissolve one or more keratin-based adhesive bonds, methods for preparing the one or more compositions, and methods for applying the one or more compositions. The one or more compositions may be embodied as, used as part of, and/or applied to numerous different technologies, products, and/or processes that have a need to at least partially remove and/or dissolve keratin-based adhesives. Example technologies, products, and/or processes may include hair care, or beauty, products; cosmetic technologies; medical adhesive systems; biomedical engineering systems; and the like. In general, the aspects described herein may generally improve the ways in which keratin-based adhesives may be dissolved and/or removed in any bonding application.

Keratin is a fibrous protein naturally found in many structures including hair, nails, outer layer of skin, and the like. Today, keratin may be used as a part of a keratin-based adhesive in which the keratin-based adhesive can be melted, then cooled to a hardened state which results in a bond between at least two surfaces (e.g., bond another surface to hair, skin, nails, and the like). These keratin-based adhesive bonds may include structures and materials such as silicone, plasticizers, co-polyamides, and various polymers.

Keratin-based adhesives may take various forms including glues, gels, beads, sticks, liquids, films, coatings, dressings, materials, and the like. The exact form of keratin-based adhesive may depend on the bonding application. For example, applications where keratin bond hair extensions are bonded to a person's hair may use keratin gel beads, keratin glue beads, keratin glue sticks, or some other keratin-derived adhesive. Applications where keratin-based adhesives are used as part of a medical adhesive system may use keratin-derived or keratin coated bonding materials, films, dressings, and the like. Applications where keratin-based adhesives are used as part of a biomedical engineering system may use keratin-derived or keratin coatings or other type of keratin-based adhesive disposed between two layers of a biomedical engineering system.

To ease the discussion, many examples of the aspects described herein will be directed to one or more compositions that may be prepared and applied such that keratin bond hair extensions can be removed from a person's hair. However, in view of the above, aspects described herein may apply to any bonding application in which keratin-based adhesives are to be dissolved and/or removed. In this way, the aspects described below, or variations thereof, may be used in different bonding applications than the below described examples of removing keratin bond hair extensions from a person's hair. For example, aspects described below, or variations thereof, may be used, or modified for use, as part of various medical adhesive systems to remove and/or reposition a prosthetic that is adhered using keratin-based adhesives, to remove wound dressings that are adhered using a keratin-based adhesive, and to remove surgical materials that are adhered using a keratin-based adhesive. As a further example, aspects described below, or variations thereof, may be used, or modified for use, as part of various cosmetic applications to remove films or coatings that use a keratin-based adhesive.

Additionally and as mentioned above, many examples of the aspects described herein will be directed to removing a keratin bond hair extension from a person's hair. The phrase “keratin bond hair extension” is used for clarity and simplicity and refers to any hair extension that may be, or is currently, bonded to a person's hair using a keratin-based adhesive. Such hair extensions may be interchangeably referred to as a keratin bond hair extension, a fusion hair extension, a keratin-bonded hair extension, and the like.

Moreover and as mentioned above, many examples of the aspects described herein will be directed to a composition that at least partially removes and/or dissolves keratin-based adhesives. The term composition is used for clarity and simplicity. Such a composition may be interchangeably referred to as keratin bond remover, a keratin bond dissolver, a dissolving agent, a removing agent, and the like.

By using various implementations of the composition described herein, the removal of keratin bond hair extensions may be improved over existing processes in many ways. For example, applying a composition described herein may result in keratin bond hair extensions being removed from a person's hair in less time and/or in a less labor intensive fashion as compared to existing processes of removing keratin bond hair extensions. As will be described below, removing a keratin bond hair extension from a person's hair may be performed by the person themselves, without assistance from another person, within a few minutes (e.g., within 1.5 to 3 minutes), and by the user of the person's fingers and/or hands (e.g., without the use of difficult tools, such as pliers, to break and pull on keratin-based adhesive bonds). As another example, a composition described herein may be formulated with, or otherwise include, ingredients that are non-toxic, or otherwise less toxic, and/or biodegradable as compared to ingredients used as part of existing processes of removing keratin bond hair extensions (e.g., the composition may not use or otherwise include ethyl alcohol, mineral oils, and the like). As yet another example, a composition described herein may be formulated with, or otherwise include, ingredients that are plant-derived, sustainable and/or renewable as compared to ingredients used as part of existing processes of removing keratin bond hair extensions. Additional improvements will be discussed and apparent based on the examples and discussion of this disclosure.

A composition that at least partially removes and/or dissolved keratin-based adhesive bonds may include one or more active ingredients, one or more supporting ingredients, one or more optional additives, and one or more base ingredients. Some or all of the active ingredients, supporting ingredients, optional additives, and base ingredients may be selected for their synergistic functionality. The active ingredients, supporting ingredients, optional additives, and base ingredients may be interchangeably referred herein as “components”, “ingredients”, “agents”, and “additives”.

The one or more active ingredients may be about 5-13% by weight of the composition. In some embodiments, the active ingredients may include dimethyl sulfoxide (DMSO) and/or one or more proteolytic enzymes. DMSO may act as a solvent that disrupts hydrogen bonds within the matrix of the keratin-based adhesive, which causes softening of the keratin-based adhesive. Put another way, DMSO may alter the keratin-based adhesive's internal hydrogen bond network, reducing its structural rigidity. Further, in arrangements where one or more proteolytic enzymes are included along with DMSO in the composition, DMSO may facilitate enzymatic access by enhancing the transport of proteolytic enzymes into the keratin-based adhesive's internal structure for uniform degradation. Put another way, DMSO may act as a carrier molecule, enabling proteolytic enzymes to reach deeper layers of the keratin-based adhesive. The one or more proteolytic enzymes may include papain, bromelain, or a blend of papain and bromelain. Papain may be derived from papaya (e.g., Carica papaya) and, as a cysteine protease, may hydrolyze peptide bonds in keratin, targeting disulfide cross-links to disassemble adhesive structures. Bromelain may be derived from pineapple (e.g., Ananas comosus) and, as a complementary protease, may broaden the spectrum of keratin degradation by breaking non-disulfide peptide linkages. In arrangements where papain is included along with bromelain in the composition, the presence of bromelain may enhance the efficiency of papain, and the combined action of papain and bromelain may increase the spectrum of keratin degradation and/or may ensure complete dissolution. In some arrangements, the one or more active ingredients may include both DMSO (e.g., at about 2-5% by weight of the composition) and one or more proteolytic enzymes (e.g., at about 3-8% by weight of the composition). In other arrangements, DMSO may be omitted and the one or more active ingredients may include one or more proteolytic enzymes (e.g., at about 5-7.5% by weight of the composition).

The one or more supporting ingredients may be about 42.25-55.65% by weight of the composition. The one or more supporting ingredients may include some or all of the following: one or more solvents for applying an active ingredient to keratin-based adhesive bonds, one or more emulsifiers for at least partially homogenizing the composition, one or more penetration enhancers for applying an active ingredient to keratin-based adhesive bonds; one or more activators for applying an active ingredient to keratin-based adhesive bonds; one or more pH adjusters for adjusting pH to a level suitable for application to human hair and/or scalp (e.g., a pH range around 5.2-5.8 in some arrangements); one or more stabilizers for the active ingredient; one or more conditioning oils for human hair; one or more repair agents for human hair; one or more humectants for attracting water to human scalp; one or more emollients for softening or smoothing human hair and/or scalp; one or more other moisturizing agents for human hair and/or human scalp, and the like. There are a wide variety of ingredients that can be used as the above-mentioned solvents, penetration enhancers, activators, pH adjusters, stabilizers, conditioning oils, repair agents, humectants, emollients, other moisturizing agent, and the like. Further, some ingredients may provide more than a single function (e.g., an ingredient may be both an emollient and a solvent). For simplicity and clarity, the remaining discussion of the one or more supporting ingredients will provide a listing of different example ingredients, examples of the functions they provide to the composition, and example percentages by weight that may be suitable in a composition. Following the listing are a few particular examples of arrangements for supporting ingredients that are suitable for a composition that at least partially removes and/or dissolves keratin-based adhesive bonds. The examples of the functions they provide may be only some of the functions provided by an ingredient and the additional functions would be apparent based on the nature of the ingredient itself.

One example of a supporting ingredient may be isopropyl myristate at about 10%-15% by weight of the composition. Isopropyl myristate may function as a solvent that assists in dissolving residual keratin fragments and assists in providing a residue-free finish. Isopropyl myristate may function as an emollient to soften and/or smooth human hair and/or human scalp.

Another example of a supporting ingredient may be coconut-derived fatty alcohols at about 5% by weight of the composition. Coconut-derived fatty alcohols may function as a solvent that assists in the softening of keratin-based adhesive bonds by reducing bond rigidity, which may complement actions of any of the active ingredients.

Yet another example of a supporting ingredient may be one or more plant-based oils at about 5-10% by weight of the composition. A plant-based oil may include argan oil, jojoba oil, or a blend thereof. Argan oil may provide omega fatty acids and vitamin E, which restore moisture and improve hair elasticity. Further, argan oil may function as an emollient, other moisturizing agent, and/or conditioning oil that provides hydration to hair and/or scalp, may repair damage to hair and/or scalp, and may, via its antioxidant properties, protect hair from oxidative stress during the hair extension removal process. Jojoba oil may mimic natural sebum, maintaining the scalp's protective barrier. Further, Jojoba oil may function as a moisturizing agent and/or conditioning oil by mimicking natural sebum to maintain scalp moisture and elasticity. Depending on formulation goals, a further benefit of plant-based oils may that they can be derived from renewable and/or biodegradable sources.

Another example of a supporting ingredient may be hydrolyzed silk protein at about 2-3% by weight of the composition. Hydrolyzed silk proteins may adhere to damaged cuticles thereby reinforcing the hair structure and/or may create a film that locks in moisture, reducing frizz and enhancing shine. Hydrolyzed silk protein may be plant-based silk proteins, such as quinoa-derived silk proteins, rice-derived silk proteins, or a blend thereof. Hydrolyzed silk protein may function as a repair agent by repairing micro-damage in the cuticle of hair, by binding to negatively charged sites, and/or by leaving hair stronger and shinier.

Yet another example of a supporting ingredient may be citric acid at about 0.1-0.3% by weight of the composition. The citric acid may be anhydrous and may function as a pH adjuster that is used to adjust the pH level based on applying an active ingredient to human hair and/or scalp. In some arrangements, the pH level may be targeted for about 4.5-5.5, as a way to optimize enzymatic activity of proteolytic enzymes and to reduce irritation risk of the scalp. Some further acceptable range may be built in for variations among batches of the composition (e.g., if a pH target is set for 5.5, the acceptable range may be about 5.2-5.8 pH).

Another example of a supporting ingredient may be L-cysteine hydrochloride (HCL) at about 0.10% by weight of the composition. L-cysteine HCL may function as an activator for one or more of the active ingredients (e.g., an activator for the one or more proteolytic enzymes).

Yet another example of a supporting ingredient may be sodium phytate at about 0.15% by weight of the composition. Sodium phytate may function as a stabilizer for an active ingredient. For example, sodium physate may function as a chelator for proteolytic enzyme stability.

Another example of a supporting ingredient may be dimethyl isosorbide at about 7% by weight of the composition. Dimethyl isosorbide may function as a penetration enhancer for an active ingredient. For example, dimethyl isosorbide may soften keratin-based bonds to allow a proteolytic enzyme to penetrate further into the bonded area.

Yet another example of a supporting ingredient may be ethoxydiglycol (e.g., TRANSCUTOL P) at about 1.5% by weight of the composition. Ethoxydiglycol may function as a penetration enhancer for an active ingredient and/or a solvent for applying an active ingredient to keratin-based adhesive bonds.

Another example of a supporting ingredient may be propylene carbonate at about 3% by weight of the composition. Propylene carbonate may function as a penetration enhancer for an active ingredient and/or a solvent for applying an active ingredient to keratin-based adhesive bonds.

Yet another example of a supporting ingredient may be C13-15 alkane (e.g., hemisqualane) at about 2% by weight of the composition. C13-15 alkane may function as a solvent for applying an active ingredient to keratin-based adhesive bonds and/or an emollient.

Another example of a supporting ingredient may be triethyl citrate at about 5-6% by weight of the composition. Triethyl citrate may function as a solvent for applying an active ingredient to keratin-based adhesive bonds and/or an emollient.

Yet another example of a supporting ingredient may be triacetin (e.g., glycerol triacetate) at about 2.5% by weight of the composition. Triacetin may function as a solvent for applying an active ingredient to keratin-based adhesive bonds and/or an emollient.

Another example of a supporting ingredient may be decyl glucoside (e.g., 50% a.i.) at about 1.5% by weight of the composition. Decyl glucoside may function as an emulsifier and/or a surfactant.

Yet another example of a supporting ingredient may be caprylyl/capryl glucoside (e.g., 60% a.i.) at about 0.75% by weight of the composition. caprylyl/capryl glucoside may function as an emulsifier and/or a surfactant.

Another example of a supporting ingredient may be urea at about 2.5% by weight of the composition. Urea may function as a humectant.

Yet another example of a supporting ingredient may be glycerin at about 2.5% by weight of the composition. Glycerin may function as a humectant.

Another example of a supporting ingredient may be sodium citrate at about 0.25% by weight of the composition Sodium citrate may function as a pH adjuster that is used to adjust the pH level based on applying an active ingredient to human hair and/or scalp. As mentioned above when describing citric acid, a pH level may be targeted for about 4.5-5.5, as a way to optimize enzymatic activity of proteolytic enzymes and to reduce irritation risk of the scalp. Some further acceptable range may be built in for variations among batches of the composition (e.g., if a pH target is set for 5.5, the acceptable range may be about 5.2-5.8 pH).

As one particular example of supporting ingredients at about 42.25%-55.65% by weight of the composition, this particular example includes supporting ingredients as follows: L-cysteine HCL at about 0.10% by weight of the composition, sodium phytate at about 0.15% by weight of the composition, dimethyl isosorbide at about 7% by weight of the composition, ethoxydiglycol at about 1.5% by weight of the composition, propylene carbonate at about 3% by weight of the composition, isopropyl myristate at about 12% by weight of the composition, C13-15 alkane at about 2% by weight of the composition, triethyl citrate at about 6% by weight of the composition, triacetin at about 2.5% by weight of the composition, decyl glucoside at about 1.5% by weight of the composition, caprylyl/capryl glucoside at about 0.75% by weight of the composition, a blend of argan oil and jojoba oil at about 5% by weight of the composition, hydrolyzed silk proteins at about 2.5% by weight of the composition, urea at about 2.5% by weight of the composition, glycerin at about 2.5% by weight of the composition, citric acid at about 0.15% by weight of the composition, and sodium citrate at about 0.25% by weight of the composition.

As another particular example of supporting ingredients at about 42.25%-55.65% by weight of the composition, this additional particular example includes isopropyl myristate at about 10-15% by weight of the composition, coconut-derived fatty alcohols at about 5% by weight of the composition, one or more plant-based oils at about 5-10% by weight of the composition, hydrolyzed silk protein at about 2-3% by weight of the composition, citric acid at about 0.1-0.3% by weight of the composition, and a selection of any number of other above-mentioned supporting ingredients to bring the total amount of supporting ingredients to or within the 42.25-55.65% range by weight of the composition. The selection may be based on formulation goals and/or a target for the total amount of supporting ingredients. As one example selection, including L-cysteine HCL at about 0.1% by weight of the composition, sodium phytate at about 0.15% by weight of the composition, dimethyl isosorbide at about 7% by weight of the composition, ethoxydiglycol at about 1.5% by weight of the composition, propylene carbonate at about 3% by weight of the composition, C13-15 alkane at about 2% by weight of the composition, and triethyl citrate at about 6% by weight of the composition, and triacetin at about 2.5% by weight of the composition would bring the supporting ingredients to or within the 42.25-55.65% range by weight of the composition

The one or more optional additives may be about 0.6-6% by weight of the composition. The one or more optional additives may include one or more thickeners for the composition, one or more preservatives for the composition and/or one or more soothing agents for the composition. The range by weight of 0.6-6% by weight of the one or more optional additives may be broken down further such that the one or more optional additives include thickeners at about 0.0-5.65% by weight, preservatives at about 0.1-0.8% by weight, and soothing agents at about 0.25%-1% by weight. Thickeners (e.g., 0.0-5.65% by weight) may include cetyl alcohol, xantham gum, hydroxyethylcellulose (HEC), and the like. Preservatives (e.g., 0.1-0.8% by weight) may include phenoxyethanol or some other suitable preservative that extends the stability of the composition. Soothing agents (e.g., 0.25-1% by weight) may include bisabolol, lavender essential oil, aloe, or some other suitable soothing agent that provides an aromatic to the composition and/or an anti-inflammation effect to the composition. An optional additive may provide more than one function for the composition. For example, cetyl alcohol (e.g., a fatty alcohol as opposed to an ethyl alcohol) may also act as an emulsifier for at least partially homogenizing the composition and xanthan gum may also act as a preservative by stabilizing the composition. As one example arrangement where the one or more optional additives are about 5.35% by weight of the composition, the one or more optional additives may include cetyl alcohol at about 3.5% by weight, HEC at about 0.60% by weight, xantham gum at about 0.2% by weight, phenoxyethanol at about 0.80% by weight, and bisabolol at about 0.25% by weight. As another example arrangement where the one or more optional additives are about 0.6-1.2% by weight of the composition and include no thickeners, the one or more optional additives may include lavender essential oil at about 0.5-1% by weight and phenoxyethanol at about 0.1-0.2% by weight.

Whether a specific ingredient is considered a supporting ingredient or an optional additive may vary from composition to composition and may depend on the bonding application the composition is intended for. The discussion above providing examples of particular supporting ingredients and particular optional ingredient are for clarity and simplicity. Indeed, the discussion above is intended to show supporting ingredients that provide direct benefits to removing keratin bond hair extensions from a person's hair (e.g., one or more solvents for applying active ingredient(s) to keratin-based adhesive bonds, emulsifiers for at least partially homogenizing the composition, penetration enhancers for applying active ingredient(s) to keratin-based adhesive bonds, activators for active ingredients, pH adjusters for setting a pH level based on applying active ingredient(s) to human hair/scalp, stabilizers for the active agent, conditioning oils for human hair, repair agents for human hair, humectants for attracting water to human scalp, emollients for softening or smoothing human scalp, and the like). Further, the discussion above is intended to show optional additives that provide indirect benefits to removing keratin bond hair extensions from a person's hair (e.g., thickeners that adjust the viscosity of the composition, preservatives that extend the stability of the composition, and soothing agents that provide additional functions like aromatic and anti-inflammatory functions. However, including all supporting ingredients and optional additives under one of those categories, or an alternative category (e.g., inactive ingredients or functional ingredients), would not change the functions of those ingredients. Each ingredient provides a function to the composition and the above examples are selected for clarity and simplicity.

The base ingredient may be adjusted to balance the other components of the composition and may be at most 38.75% by weight of the composition. From this maximum amount, the remaining ingredients (e.g., the active ingredients, supporting ingredients, and optional additives) may be at least 61.25% by weight of the composition, which allows for all ingredients of the composition to be 100% by weight of the composition with a maximum amount of base ingredient. Base ingredients may include various types of water and/or oil. For clarity, the examples of this disclosure will use a basic ingredient of water suitable for cosmetic use. Water suitable for cosmetic use may include water free of toxins, free of pollutants, free of microbes, low in calcium, and low in heavy metals (lead, copper, etc.). The Water suitable for cosmetic use includes distilled water, deionized water, demineralized water, and purified water. The exact amounts of base ingredient and remaining ingredients in the composition may depend on particular formulation goals, which can be varied. Some example formulation goals include a target pH level for the composition (e.g., a target pH of 5.5 or a quality control pH range of about 5.2-5.8 for the composition); a target viscosity for the composition (e.g., thicker viscosity may cause more thickeners and less water to be in the composition and a thinner viscosity may cause fewer supportive agents and more water to be in the composition); how aromatic the composition should be (e.g., adding more aromatic components may lower the amount of water by weight in the composition); how anti-inflammatory the composition should be (e.g., adding an additional 3% by weight of aloe may remove 3% by weight of water in the formulation while maintaining all remaining ingredients at the same percentage by weight); and the like. The above are only a small subset of example formulation goals that may be used when formulating a composition according to aspects described herein.

A composition may remove and/or dissolve keratin-based adhesive bonds via solvent penetration and enzymatic degradation that is activated by heat and/or massaging. Heating may include applying heat, via heat lamp or blow dryer, until the keratin-based adhesive bond reaches about 50-60 degrees Celsius. For example, in formulations that include DMSO and/or isopropyl myristate, among other ingredients, solvent penetration may be achieved by applying heat and/or massaging such that hydrogen bonds break down and ionic interactions within the keratin adhesive matrix are disrupted, softening its structure, and enabling enzymatic penetration. As another example, in formulations that include papain and/or bromelain, among other ingredients, enzymatic degradation may be achieved by applying heat and/or massaging such that specific peptide bonds within keratin are hydrolyzed, the keratin-based adhesive bonds are broken into soluble fragments for easy removal. In formulations that include both papain and bromelain, enzymatic degradation may be further achieved by applying heat and/or massaging such that various keratin crosslinks are degraded.

A composition may treat human hair and/or scalp by conditioning and/or protection functions. For example, in formulations that include plant-based oils and/or hydrolyzed silk protein, conditioning and/or protection functions may be achieved by forming a protective layer on natural hair, preventing dryness, brittleness, and scalp irritation. Further, by achieving the conditioning and/or protection functions, hair integrity may be preserved while promoting a smooth, residue-free finish.

Various formulations of the composition described herein may be compliant with Federal Drug Administration (FDA) regulations for cosmetic products, including safety and efficacy regulations. Further, once packaged, the package may further comply with any labeling regulation. Various formulations of the composition described herein may be compliant with European Union (EU) cosmetic regulation (e.g., EC No 1223/2009), including compliance with ingredient sourcing, testing, and biodegradability. Various formulations of the composition described herein may be compliant with one or more eco-certifications. Some formulations may include ingredients that are certified biodegradable and sustainably sourced (e.g., plant oils, fatty alcohols, and enzymes may be derived from renewable resources). Various formulations may satisfy one or more OECD biodegradability standards (e.g., successfully completing a standardized test finding all ingredients degrade safely within 30 days in aerobic conditions).

Various formulations of the composition may be tested to measure various performance characteristics of the composition. Various formulations may have been tested on a range of skin types, including sensitive skin, with no reported adverse reactions. For example, under controlled circumstances and using a patch applied to 100 participants with varying skin types, including sensitive skin, no adverse reactions were reported. Further, various formulations may score at 0.05 on the irritation index scale, indicating negligible irritation potential. Various formulations may have demonstrated complete keratin bond dissolution within 1.5-3 minutes, outperforming traditional keratin-based adhesive removers. Various formulations may demonstrate reduced adhesive strength by 85% within the first minute under controlled laboratory testing. Various formulations may, under scanning electron microscopy (SEM), show no residual adhesive on hair strands post-treatment. Various formulations may have demonstrated complete bond removal without leaving residue, eliminating the need for additional cleaning steps. Various formulations may have been put under a hair integrity test with results showing a 92% reduction in hair breakage compared to acetone-based keratin-based adhesive bond removers.

The composition may be packaged in various types of packages and offerings. For example, the composition may be packaged in bottles or otherwise packaged in a way that allows for user convenience, sustainability, and professional efficacy. The packaging may be made from recyclable glass or Bisphenol A (BPA-free) plastic. The packaging may be in different sizes for different uses (e.g., a 4 oz or larger bottle for salon use or a 2 oz bottle for travel or at-home use). The packaging may include spray nozzles for fast, wide-area application and/or may include a precision dropper for targeted application. The packaging may include tamper-proof mechanisms in an attempt to ensure product integrity and safety. The packaging may include Quick-Response (QR) code integration as a way to provide users with access to instructional videos, usage guides, and/or troubleshooting tips.

### Methods of Preparation

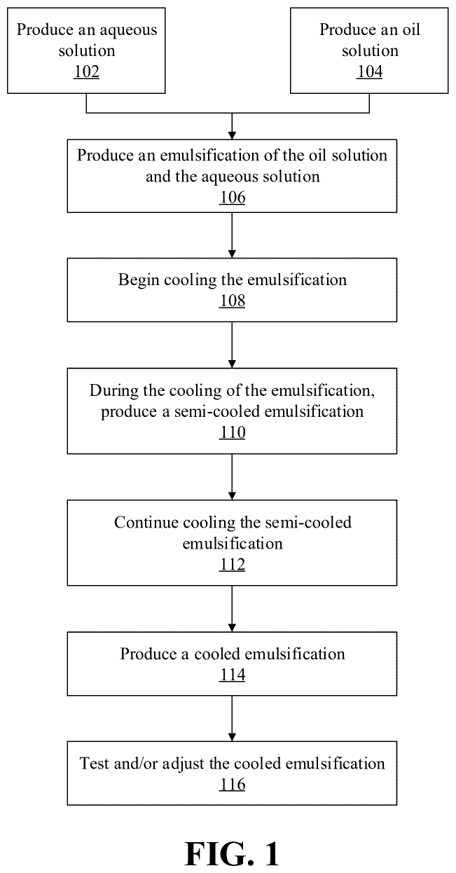

FIG. 1 depicts an example method of making a composition to assist in removing and/or dissolving one or more keratin-based adhesive bonds in accordance with one or more embodiments described herein. While the example method depicted in FIG. 1 provides steps for making various compositions described herein, some details will be with particular respect to a composition according to the formulation of Table 1. According to some aspects, techniques other than those described herein may be used to prepare other formulations different than those of FIG. 1 and Table 1. The formulation of Table 1, which includes only a blend of papain and bromelain as an active ingredient, among other ingredients, is as follows.

TABLE 1

| | Percentage (%) weight |
|---|---|
| Component | of the composition |
| Blend of papain and | 6.5% |
| bromelain | |
| L-cysteine HCL | 0.10% |
| Sodium phytate | 0.15% |
| Dimethyl isosorbide | 7% |
| Ethoxydiglycol | 1.5% |
| Propylene carbonate | 3% |
| Isopropyl myristate | 12% |
| C13-15 alkane | 2% |
| Triethyl citrate | 6% |
| Triacetin | 2.5% |
| Decyl glucoside | 1.5% |
| caprylyl/capryl | 0.75% |
| glucoside | |
| A blend of argan oil | 5% |
| and jojoba oil | |
| Hydrolyzed silk protein | 2.5% |
| Urea | 2.5% |
| Glycerin | 2.5% |
| Citric acid | 0.15% |
| Sodium citrate | 0.25% |
| Cetyl alcohol | 3.5% |
| Hydroxyethylcellulose | 0.6% |
| Xantham gum | 0.2% |
| Phenoxyethanol | 0.8% |
| Bisabolol | 0.25% |
| Deionized water | 38.75% |
| TOTAL | 100% |

At step 102, an aqueous solution may be produced. The aqueous solution may be produced by mixing at least a portion of the deionized water, glycerin, urea, sodium phytate together; then mixing (e.g., via sprinkling) in the hydroxyethylcellulose, xanthan gum, and hydrolyzed silk protein; then heating to about 70 degrees Celcius; and then adding the citric acid and sodium citrate.

At step 104, an oil solution may be produced. The oil solution may be produced by heating the cetyl alcohol until it melts and is heated to at or around 70 degrees Celcius; then adding the isopropyl myristate, C13-15 alkane, triethyl citrate, triacetin, the blend of argan oil and jojoba oil, and bisabolol; then mixing (e.g., until clear).

Steps 102 and 104 may be reversed in order, performed in parallel, or performed sequentially. Before proceeding to step 106, both the oil solution and the aqueous solution must be produced and separate from each other.

At step 106, an emulsification of the oil solution and the aqueous solution may be produced. To produce the emulsification, while both the oil solution and the aqueous solution are heated to about 70 degrees Celcius, the oil solution may be added to the aqueous solution and then mixed, or otherwise allowed, to homogenize.

At step 108, the emulsification may begin to cool.

At step 110, a semi-cooled emulsification may be produced. To produce the semi-cooled emulsification, when the emulsification has cooled to about 45 degrees Celsius or lower, add dimethyl isosorbide, propylene carbonate; then pre-dilute both the decyl glucoside and caprylyl/capryl glucoside with at least a portion of the deionized water (e.g., both the decyl glucoside and caprylyl/capryl glucoside may be pre-diluted a 1:1 ratio with deionized water, where the deionized water is about 30-45 degrees Celsius); and then add the pre-diluted decyl glucoside and pre-diluted caprylyl/capryl glucoside in a slow fashion to the emulsification. In a slow fashion may mean that the pre-diluted glucosides are added at a rate that prevents or avoids clumping of the glucosides and/or prevents or avoids foaming, overflowing, and separation of the emulsification.

At step 112, the semi-cooled emulsification may continue to cool.

At step 114, a cooled emulsification may be produced. To produce the cooled emulsification, when the semi-cooled emulsification has cooled to about 30 degrees Celsius or lower, add the ethoxydiglycol and phenoxyethanol to the semi-cooled emulsification; then add the blend of papain and bromelain and the L-cysteine HCL; and then mix the semi-cooled emulsification.

At step 116, the cooled emulsification may be tested and/or adjusted. Testing and/or adjusting the cooled emulsification may include testing the pH level of the cooled emulsification (e.g., ensure the cooled emulsification has a pH of around 5.5, or within a range of 5.2-5.8 pH); adding any deionized water needed to adjust the pH, if needed; and de-air the cooled emulsification (e.g., let the cooled emulsification rest and settle). Once step 116 is complete (e.g., the cooled emulsification has a pH of around 5.5 or within a range of 5.2-5.8 pH), the making of the composition may be considered complete and the cooled emulsification may be packaged for use (e.g., filled into the desired packaging), and/or allowed to be used to at least partially remove and/or dissolve keratin-based adhesive bonds according to the aspects described herein.

### Methods of Application

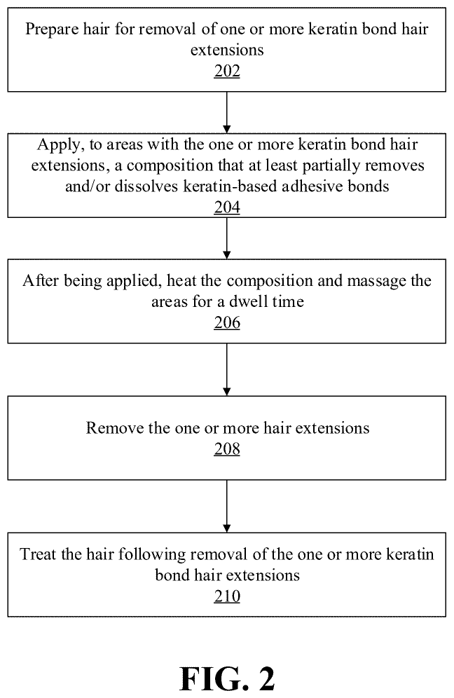

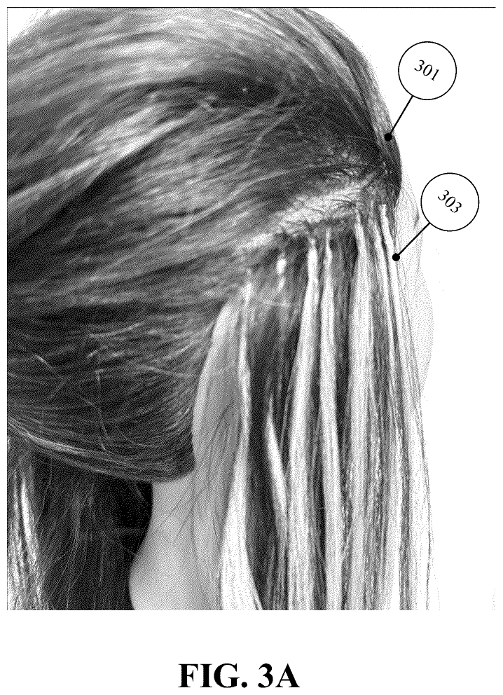

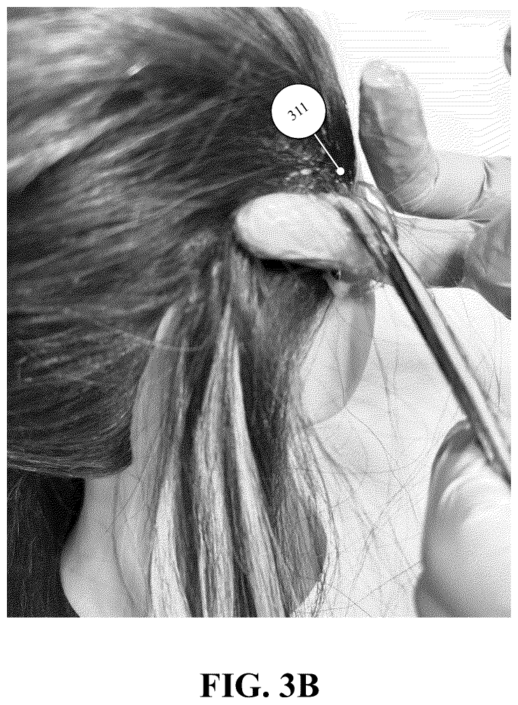

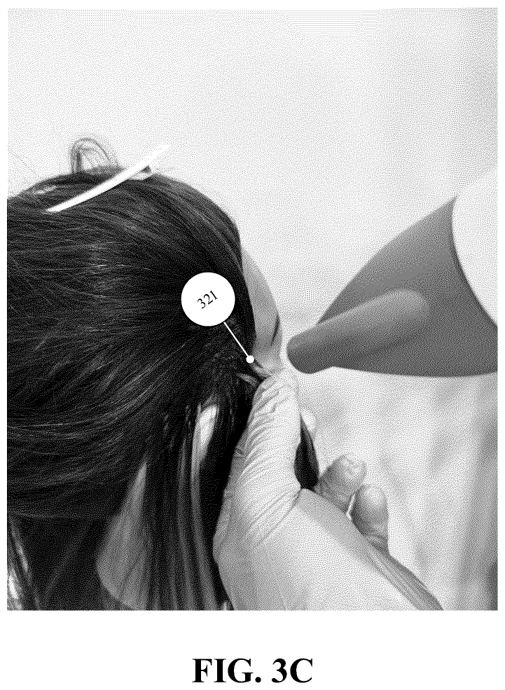

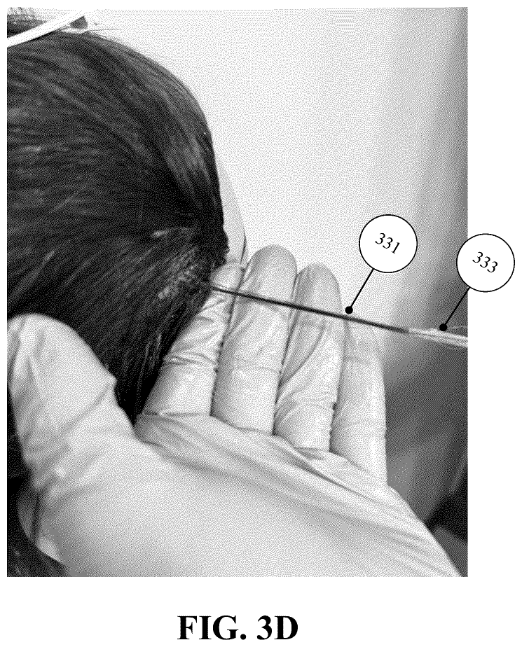

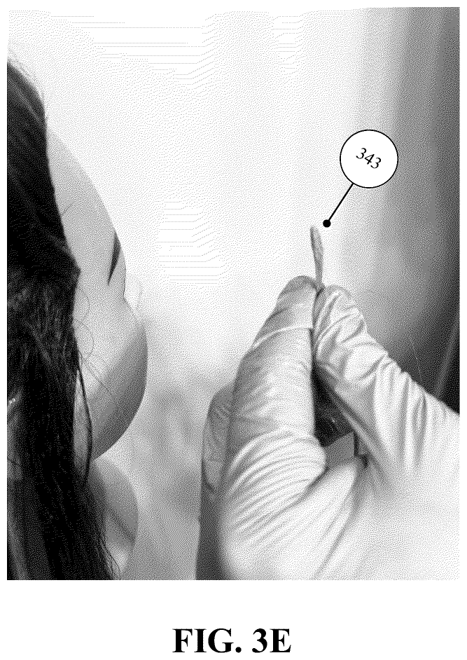

FIG. 2 depicts an example method of applying a composition to assist in removing and/or dissolving one or more keratin-based adhesive bonds in accordance with one or more embodiments described herein. While the example method depicted in FIG. 2 provides steps for applying various compositions described herein, some details will be with particular respect to FIGS. 3A-3D, which depict example illustrations of applying a composition to assist in removing and/or dissolving one or more keratin-based adhesive bonds. In particular, FIGS. 3A-3E depict examples where the formulation of Table 1, which includes only a blend of papain and bromelain as an active ingredient, among other ingredients, is applied to remove a keratin-bond hair extension. The depictions of FIGS. 3A-3E provide examples where a single person removes a keratin bond hair extension from hair (e.g., without requiring a second person). It will be apparent based on FIGS. 3A-3E that the keratin bond hair extension could be removed by a single person from that single person's hair. Further, the depictions of FIGS. 3A-3E provide examples where a single person uses their fingers and/or hand to remove a keratin bond hair extension. For clarity, FIGS. 3A-3E provide examples where keratin bond hair extensions (e.g., extension 303 of FIG. 3A) are contrasted from the hair (e.g., hair 301 of FIG. 3A) the extensions are bonded to and/or removed from. Indeed, FIGS. 3A-3E provide examples where keratin bond hair extensions includes blond, or lighter colored, extension hair; and where the hair such extensions are bonded to and/or removed from include brown, or darker colored, hair.

According to some aspects, techniques other than those described herein may be used to apply various formulations of the compositions described herein. According to some aspects, techniques other than those described herein may be used to prepare other formulations different than those of FIG. 2, FIGS. 3A-3E, and Table 1. Indeed, a formulation using DMSO along with a blend of papain and bromelain may be applied in a similar fashion as described in connection with FIG. 2, FIGS. 3A-3E, and Table 1, and may exhibit similar results as described in connection with FIG. 2, FIGS. 3A-3E, and Table 1.

At step 202, the hair may be prepared for removal of one or more keratin bond hair extensions. To prepare the hair, portions of the hair may be sectioned, or bound, to isolate any area where the one or more keratin bond hair extensions are bonded to the hair. The sectioning, or binding, may be performed by a sectioning clip or some other hair clip suitable to isolate any area where the one or more keratin bond hair extensions are bonded to the hair. Additionally, to prepare the hair, any areas where the one or more keratin bond hair extensions are bonded to hair may compressed to aide in removing and/or dissolving the keratin-based adhesive bonds. For example, a set of pliers may be placed such that one tooth of the plier is above an area where the one or more keratin bond hair extensions are bonded to the hair and the other tooth of the plier is below the area. Once placed, the pliers may be closed such that some force is placed on the area and the keratin-based adhesive bonds at the area are compressed (e.g., no pulling on the hair or the keratin-based adhesive bonds is performed). FIG. 3A depicts an example where a keratin bond hair extension 303 is isolated from hair 301 to prepare for its removal. In some arrangements, step 202 may be optional (e.g., hair may not be prepared and instead, the method may proceed directly to step 204). Whether step 202 is or is not performed may depend on the comfort level of the person removing the one or more hair extensions (e.g., the person may always opt to prepare the hair) and/or the particular formulation of the composition (e.g., some formulations may benefit the compressing with pliers of the keratin-based adhesive bonds, while other formulations may not require the compressing). For purposes of FIG. 3A, assume the compressing of the keratin-based adhesive bonds has been performed as part of the preparation of the hair.

At step 204, a composition that at least partially removes and/or dissolves keratin-based adhesive bonds may be applied to areas with the one or more hair extensions. For example, the composition may be applied using fingers, hands, a spray nozzle, a precision dropper, and/or any combination thereof. Further, the composition may be applied broadly (e.g., to an area that includes one or more keratin-based adhesive bonds and another area that does not include keratin-based adhesive bonds) and/or the composition may be applied in a targeted fashion (e.g., as best to one's ability, only to an area that includes keratin-based adhesive bonds). FIG. 3B depicts an example where a composition is being applied at 311 using hands and/or fingers (e.g., the composition may be applied and manipulated, via two fingers, around an area that includes one or more keratin-based adhesive bonds).

At step 206, the composition, after being applied to an area that includes one or more keratin-based adhesive bonds, may be heated and/or massaged for a dwell time. The heating, massaging, and/or dwell time allow the composition to remove and/or dissolve the keratin-based adhesive bonds at the area. In some arrangements, the composition may be heated to about 50-60 degrees Celsius. The massaging may be performed by fingers and/or hands (e.g., similar to the massaging shown in FIG. 3B at 311 when applying the composition). The dwell time required may be between 1.5 and 3 minutes depending on formulation of the composition. FIG. 3C depicts an example where an area 321 that has been applied with a composition is both being heated by a blow dryer and massaged by fingers. For purposes of the examples of FIGS. 3A-3C, the dwell time may be considered 1.5 minutes.

At step 208, the one or more keratin bond hair extensions may be removed. The removal may be performed using fingers and/or hands, or some other suitable tool for removing the one or more keratin bond hair extensions (e.g., a fine-tooth comb). The tool chosen to remove the one or more keratin bond hair extensions may depend on the comfort level and/or experience of a person performing this step (e.g., the person performing the removing may use their fingers, hands, a comb, pliers, or some combination of tools of their own selection). FIG. 3D depicts an example where a keratin bond hair extension 333 is being removed from hair 331 by one hand (not shown) pulling on the keratin bond hair extension 333. The second, non-pulling, hand is shown behind the hair 331 to provide contrast and highlight that no breaking of the hair 331 is apparent as the keratin bond hair extension 333 is being pulled on and removed from the hair 331. FIG. 3E further depicts the keratin bond hair extension 343 after it has been removed from the hair (e.g., hair 331 of FIG. 3D). As depicted in FIG. 3E, the keratin bond hair extension 343 does not include any strands, or broken pieces, from the hair the keratin bond hair extension 343 was previously bonded to.

In view of steps 204-208 and the examples of FIGS. 3A-3E, it can be seen that some formulations of the composition, after being applied, enable a single person to remove the one or more keratin bond hair extensions with their fingers and/or hands from their own hair.

At step 210, the hair may be treated following removal of the one or more keratin bond hair extensions. In some arrangements, the treatment following removal of the one or more keratin bond hair extensions may include washing the hair with a sulfate-free shampoo and/or with a keratin-repairing conditioner. In some arrangements, step 210 may be optional (e.g., hair may not be treated following removal of the one or more keratin bond hair extensions if, for example, no residue is felt and/or the hair appears healthy, and the method may end). Whether step 210 is or is not performed may depend on the formulation that was applied (e.g., a formulation may remove and/or dissolve keratin-based adhesive bonds so fully that the hair may not require treatment; a formulation may treat the hair sufficiently with moisturizers that the hair may not require treatment). Whether step 210 is or is not performed may depend on the comfort level of the person removing the one or more hair extensions (e.g., the person may always opt to treat the hair).

### Statements

The following statements provide additional examples of some formulations of the composition described herein.

Statement 1, a composition comprising: dimethyl sulfoxide (DMSO) (2-5%): a solvent that penetrates and disrupts hydrogen bonds within keratin-based adhesives; isopropyl myristate (10-15%): a lightweight solvent that dissolves adhesive residues and provides a residue-free finish; proteolytic enzymes (5-8%): selected from papain, bromelain, or combinations thereof, for hydrolyzing peptide bonds in keratin; coconut-derived fatty alcohols (5%): for softening keratin adhesive bonds to facilitate removal; plant-based oils (5-10%): including argan oil and jojoba oil, for conditioning and protecting natural hair and scalp; hydrolyzed silk protein (2-3%): to repair and strengthen hair fibers during adhesive removal; and citric acid (0.1-0.3%): as a pH adjuster to maintain the composition's pH between 4.5 and 5.5 for optimal enzymatic activity and scalp compatibility.

Statement 2, the composition of statement 1, wherein the ingredients are: biodegradable, sustainably sourced, and free from acetone, alcohol, mineral oils, and other harsh chemicals.

Statement 3, the composition of statement 1, wherein keratin bonds dissolve completely within 1-2 minutes after application, without the need for excessive mechanical force or the use of heat.

Statement 4, a method of removing keratin-bonded hair extensions using the composition of statement 1, comprising: applying the composition directly to keratin bonds, ensuring complete coverage; allowing the composition to penetrate and activate for 1-2 minutes; and removing the hair extensions with minimal mechanical effort, such as sliding them off using a fine-tooth comb or professional pliers.

Statement 5, the method of statement 4, wherein the removal process further includes a post-removal care regimen comprising: rinsing the hair with a sulfate-free shampoo to remove residual adhesive or product; following with a keratin-repairing conditioner to restore hydration, strength, and elasticity to the hair.

Statement 6, a method of dissolving keratin-based adhesives in medical applications, using the composition of statement 1, for removing keratin-based prosthetic adhesives, dissolving keratin-coated wound dressings, or cleaning surgical instruments or devices coated with keratin-based residues.

Statement 7, the method of statement 6, wherein the composition minimizes patient discomfort by being hypoallergenic, dermatologically tested, and suitable for sensitive skin.

Statement 8, a method of removing keratin-based adhesives from industrial or cosmetic applications, using the composition of statement 1, to dissolve: keratin films used in biomaterial coatings, residues in cosmetic formulations, or adhesive coatings on biomedical components.

Statement 9, a packaged keratin bond remover composition as described in statement 1, wherein the product is: contained in tamper-proof, recyclable bottles made from glass or Bisphenol A free (BPA-free) plastic.

Statement 10, the packaged product of statement 9, wherein the bottle is equipped with: a spray applicator for wide-area application to multiple bonds simultaneously, or a precision dropper for targeted application to individual keratin adhesive areas.

Statement 11, the packaged product of claim 9, further comprising: a user-friendly label detailing step-by-step instructions for professional and at-home use; and an integrated Quick-Response (QR) code linking to video tutorials, troubleshooting guides, and professional tips.

Statement 12, the composition of statement 1, wherein the proteolytic enzymes are stabilized with magnesium ions, calcium ions, or a pH buffer to enhance enzymatic activity and prolong shelf life.

Statement 13, the composition of statement 1, wherein the plant-based oils further include: olive oil, for additional hydration and scalp conditioning, or sunflower oil, for enhanced antioxidant and anti-inflammatory effects.

Statement 14, the composition of statement 1, wherein the formulation includes: lavender essential oil (0.5-1%) to provide a calming fragrance and reduce scalp inflammation.

Statement 15, the composition of statement 1, wherein the composition is pH-adjusted to a range of 4.8-5.2 to optimize enzymatic activity and compatibility with sensitive scalps.

Statement 16, the composition of statement 1, wherein the adhesive removal process reduces hair breakage by at least 90% compared to acetone-based removers.

Statement 17, the composition of statement 1, wherein the formulation ensures a residue-free finish, eliminating the need for additional cleaning steps post-removal.

Statement 18, the composition of statement 1, wherein the enzymatic activity remains effective at temperatures ranging from 5 degrees Celsius to 40 degrees Celsius, ensuring performance in a variety of environmental conditions.

Statement 19, the composition of statement 1, for use in hair care applications to: remove keratin-bonded hair extensions with minimal mechanical stress, and hydrate and strengthen natural hair during the removal process.

Statement 20, the composition of statement 1, for use in medical applications to: safely dissolve keratin-based adhesives on prosthetics, non-invasively remove keratin-coated wound dressings, and aid in cleaning surgical instruments coated with keratin residues.

Statement 21, the composition of statement 1, for use in cosmetic or industrial applications to: remove keratin-based coatings from biomaterials, and dissolve keratin adhesive residues in manufacturing processes.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “composition” and “compositions” used herein are equivalent, except that a single composition may refer to a single formulation for at least partially removing and/or dissolving keratin-based adhesive bonds, while plural compositions may refer to plural formulations for at least partially removing and/or dissolving keratin-based adhesive bonds. This disclosure can apply to a wide range of compositions and/or formulations.

Reference to “about” a value or parameter herein includes (and describes) variations that are directed to that value or parameter per se. For example, description referring to “about X” (e.g., about 3-5% by weight) includes description of “X” (e.g., includes 3% by weight, 5% by weight, and any amount between 3% and 5% by weight). In addition, reference to phrases “less than”, “greater than”, “at most”, “at least”, “less than or equal to”, “greater than or equal to”, or other similar phrases followed by a string of values or parameters is meant to apply the phrase to each value or parameter in the string of values or parameters. For example, a statement that a formulation has at most about 10% by weight, about 15% by weight, or about 20% by weight of a component is meant to mean that the formulation has at most about 10% by weight, at most about 15% by weight, or at most about 20% by weight of a component.

As used herein, the singular forms “a,” “an,” and “the” are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

This application discloses several numerical ranges in the text. The numerical ranges disclosed inherently support any range or value within the disclosed numerical ranges, including the endpoints, even though a precise range limitation is not stated verbatim in the specification because this disclosure can be practiced throughout the disclosed numerical ranges.

The above description is presented to enable a person skilled in the art to make and use the disclosure and is provided in the context of a particular application and its requirements. Various modifications to the preferred embodiments will be readily apparent to those skilled in the art, and the generic principles defined herein may be applied to other embodiments and applications without departing from the spirit and scope of the disclosure. Thus, this disclosure is not intended to be limited to the embodiments shown but is to be accorded the widest scope consistent with the principles and features disclosed herein.

1. A composition comprising:
one or more active ingredients that includes one or more proteolytic enzymes that at least remove and/or dissolve keratin-based adhesive bonds that are formed between hair of a person and one or more keratin bond hair extensions;
one or more supporting ingredients that includes a solvent for applying the one or more proteolytic enzymes to the keratin-based adhesive bonds, an activator for applying the one or more proteolytic enzymes to the keratin-based adhesive bonds, and a pH adjuster for setting a pH level about 5.2-5.8 based on applying the one or more proteolytic enzymes to a human hair and/or a human scalp; and
one or more base ingredients that includes deionized water.

2. The composition of claim 1, wherein the one or more active ingredients, the one or more supporting ingredients, and the one or more base ingredients are configured as a formulation that, after being applied, removes or dissolves the keratin-based adhesive bonds between the one or more keratin bond hair extensions and the hair of the person.

3. The composition of claim 1, wherein the one or more active ingredients, the one or more supporting ingredients, and the one or more base ingredients are configured as a formulation that, after being applied, enables a single person to remove the one or more keratin bond hair extensions with their fingers and/or hand from their own hair.

4. The composition of claim 1, wherein the one or more supporting ingredients includes an emulsifier for homogenizing the composition, a penetration enhancer for applying the one or more proteolytic enzymes to the keratin-based adhesive bonds, and a stabilizer for the one or more proteolytic enzymes.

5. The composition of claim 1, wherein the one or more supporting ingredients includes a conditioning oil for the human hair, a repair agent for the human hair, a humectant for attracting water to the human scalp, and an emollient for softening or smoothing the human hair and/or the human scalp.

6. The composition of claim 1, further comprising
one or more optional additives that provide an aromatic or an anti-inflammatory function to the composition.

7. The composition of claim 6, wherein:
the one or more active ingredients comprise 5-13% by weight of the composition;
the one or more supportive ingredients comprise 42.25-55.65% by weight of the composition;
the one or more optional additives comprise 0.6-6% by weight of the composition; and
the base ingredient comprises at most 38.75% by weight of the composition.

8. The composition of claim 1 wherein:
the one or more proteolytic enzymes include a blend of papain and bromelain;
the one or more supporting ingredients include L-cysteine HCL at about 0.10% by weight of the composition, sodium phytate at about 0.15% by weight of the composition, dimethyl isosorbide at about 7% by weight of the composition, ethoxydiglycol at about 1.5% by weight of the composition, propylene carbonate at about 3% by weight of the composition, isopropyl myristate at about 12% by weight of the composition, C13-15 alkane at about 2% by weight of the composition, triethyl citrate at about 6% by weight of the composition, triacetin at about 2.5% by weight of the composition, decyl glucoside at about 1.5% by weight of the composition, caprylyl/capryl glucoside at about 0.75% by weight of the composition, a blend of argan oil and jojoba oil at about 5% by weight of the composition, hydrolyzed silk proteins at about 2.5% by weight of the composition, urea at about 2.5% by weight of the composition, glycerin at about 2.5% by weight of the composition, citric acid at about 0.15% by weight of the composition, and sodium citrate at about 0.25% by weight of the composition; and
the one or more base ingredients comprise at most 38.75% by weight of the composition.

9. A composition comprising:
one or more active ingredients at about 5-13% by weight of the composition, the one or more active ingredients including one or more proteolytic enzymes that at least remove and/or dissolve keratin-based adhesive bonds that are formed between hair of a person and one or more keratin bond hair extensions;
one or more supporting ingredients at about 42.25-55.6₅% by weight of the composition, the one or more supporting ingredients including a solvent for applying the one or more proteolytic enzymes to the keratin-based adhesive bonds, an activator for applying the one or more proteolytic enzymes to the keratin-based adhesive bonds, and a pH adjuster for setting a pH level about 5.2-5.8 based on applying the one or more proteolytic enzymes to a human hair and/or a human scalp; and
one or more base ingredients at about 38.75% by weight of the composition, the one or more base ingredients including deionized water.

10. The composition of claim 9, wherein the one or more active ingredients, the one or more supporting ingredients, and the one or more base ingredients are configured as a formulation that, after being applied, removes or dissolves the keratin-based adhesive bonds between the one or more keratin bond hair extensions and the hair of the person.

11. The composition of claim 9, wherein the one or more active ingredients, the one or more supporting ingredients, and the one or more base ingredients are configured as a formulation that, after being applied, enables a single person to remove the one or more keratin bond hair extensions with their fingers and/or hand from their own hair.

12. The composition of claim 9, wherein the one or more supporting ingredients includes an emulsifier for homogenizing the composition, a penetration enhancer for applying the one or more proteolytic enzymes to the keratin-based adhesive bonds, and a stabilizer for the one or more proteolytic enzymes.

13. The composition of claim 9, wherein the one or more supporting ingredients includes a conditioning oil for the human hair; a repair agent for the human hair; a humectant for attracting water to the human scalp; and an emollient for softening or smoothing the human hair and/or the human scalp.

14. The composition of claim 9, further comprising
one or more optional additives at about 0.6-6% by weight of the composition, the one or more optional additives providing an aromatic or an anti-inflammatory function to the composition.

15. A composition comprising:
one or more active ingredients that at least remove and/or dissolve keratin-based adhesive bonds that are formed between hair of a person and one or more keratin bond hair extensions, the one or more active ingredients including dimethyl sulfoxide (DMSO) and a blend of papain and bromelain;
one or more supporting ingredients that includes a solvent for applying the one or more active ingredients to the keratin-based adhesive bonds, an activator for applying the one or more active ingredients to the keratin-based adhesive bonds, and a pH adjuster for setting a pH level about 5.2-5.8 based on applying the one or more active ingredients to a human hair and/or a human scalp; and
one or more base ingredients that includes deionized water.

16. The composition of claim 15, wherein the one or more active ingredients, the one or more supporting ingredients, and the one or more base ingredients are configured as a formulation that, after being applied, removes or dissolves the keratin-based adhesive bonds between the one or more keratin bond hair extensions and the hair of the person.

17. The composition of claim 15, wherein the one or more active ingredients, the one or more supporting ingredients, and the one or more base ingredients are configured as a formulation that, after being applied, enables a single person to remove the one or more keratin bond hair extensions with their fingers and/or hand from their own hair.

18. The composition of claim 15, wherein the one or more supporting ingredients includes an emulsifier for homogenizing the composition, a penetration enhancer for applying the one or more active ingredients to the keratin-based adhesive bonds, and a stabilizer for the one or more active ingredients.

19. The composition of claim 15, wherein the one or more supporting ingredients includes a conditioning oil for the human hair, a repair agent for the human hair, a humectant for attracting water to the human scalp, and an emollient for softening or smoothing the human hair and/or the human scalp.

20. The composition of claim 15, further comprising
one or more optional additives that provide an aromatic or an anti-inflammatory function to the composition.
